(19) United States
(12) Patent Application Publication
(10) Pub. No.: US 2021/0015752 A1
(43) Pub. Date: Jan. 21, 2021

(54) METHODS FOR PRODUCING PARTICLES OF AN ACTIVE INGREDIENT

(71) Applicant: Savior Lifetec Corporation, Miaoli County (TW)

(72) Inventors: Yan-Ming CHEN, Kaohsiung City (TW); Shih-Hsie PAN, Princeton Junction, NJ (US); Mannching Sherry KU, Thiells, NY (US); Chia-Hsin LIN, Miaoli County (TW)

(73) Assignee: Savior Lifetec Corporation, Miaoli County (TW)

(21) Appl. No.: 16/618,163

(22) PCT Filed: Jun. 14, 2018
(86) PCT No.: PCT/US2018/037431

§ 371 Date: Nov. 29, 2019

Related U.S. Application Data
(60) Provisional application No. 62/519,983, filed on Jun. 15, 2017.

(51) Int. Cl. A61K 9/16; A61K 31/519; A61K 31/375

(52) U.S. Cl. CPC A61K 9/1682; A61K 31/375; A61K 31/519

(57) ABSTRACT

Disclosed herein are particles of an active ingredient, a composition comprising the same, and a method for producing the same. The method disclosed herein comprises the steps of, (a) forming a first solution by dissolving the active ingredient in a first solvent; (b) forming the particles of the active ingredient by mixing a second solvent with the first solution to produce an emulsified solution. Moreover, the second solvent is miscible with the first solvent, but is immiscible with the first solution.

### CROSS-REFERENCES TO OTHER RELATED APPLICATION

This application claims benefit to U.S. Provisional application No. 62/519,983 filed Jun. 15, 2017, the disclosure of which is incorporated herein by reference.

### BACKGROUND OF THE INVENTION

### 1. Field of the Invention

The present disclosure relates to pharmaceutical field, and more particularly, to the particles of an active ingredient, and methods of producing the same.

### 2. Description of Related Art

One way of achieving fast released of a therapeutic agent in vivo is through the use of particles, such as nanoparticles and/or microparticles. These particles can be administered through different routes, such as oral and parenteral routes. Particles thus can be inhaled or injected (e.g., intravenously, subcutaneously or intramuscularly). Due to the easy-to-use property of particles, intensive research has been drawn to the development of improved methods of producing particles of a therapeutic agent.

Inventors of the present disclosure unexpectedly identify an improved method of producing particles, particularly, nanoparticles and microparticles, of an active agent. The present method takes advantages in the differences between solubilities of the therapeutic agent in two miscible solvent systems, so that the therapeutic agent soluble in one solvent system precipitates when another solvent is introduced therein. The particles thus produced are not only small in size, but also exhibit relatively uniform size distribution, these particles are thus suitable for use in medical formulation.

### SUMMARY

The following description presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

In one aspect, the present disclosure is directed to a method of producing particles of an active ingredient. The present method comprises the steps of:

(a) forming a first solution by dissolving the active ingredient in a first solvent;

(b) forming the particles of the active ingredient by mixing a second solvent with the first solution to produce an emulsified solution;

wherein, the second solvent is miscible with the first solvent, but is immiscible with the first solution.

According to embodiments of the present disclosure, the active ingredient may be selected from the group consisting of, a physiologically active peptide, an anti-tumor agent, an antibiotic, an anti-pyretic agent, an analgesic, an anti-inflammatory agent, an antitussive expectorant, a sedative, a muscle relaxant, an anti-epileptic, an anti-ulcer agent, an anti-depressant, an anti-allergic agent, a cardiotonic, an anti-arrhythmic agent, a vasodilator, a hypotensive diuretic, an anti-diabetic, an anti-hyperlipidemic agent, an anti-coagulant, an anti-oxidant, a hemolytic agent, an anti-tuberculosis agent, a hormone, a narcotic antagonist, a bone resorption suppressor, an osteogenesis promoter and an angiogenesis inhibitor. Further, in one embodiment, the active ingredient has a melting point, which is above 0° C.

In some embodiments, the method of present invention further comprises step (c): removing the first and second solvents from the emulsified solution to produce a powder of the particles of the active ingredient.

According to other embodiments, the method further comprises step (a-1): filtering the first solution before the step (b). In one specific embodiment, the first solution is filtered through a filter having a pore size of 0.22μm.

According to some embodiments, the first and second solvents are independently selected from the group consisting of, C₅₋₁₂ alkane, C₆₋₁₀ aromatic hydrocarbon, C₂₋₆ alkyl acetate, C₄₋₁₀ ether, C₄₋₁₀ cyclic ether, C₃₋₆ ketone, acetonitrile, and water.

Examples of the C₅₋₁₂ alkane include, but are not limited to, pentane, isopentane, hexane, cyclohexane, heptane, heptanes, octane, nonane, and etc. In one specific embodiment, the C₅₋₁₂ alkane is heptane or heptanes. Examples of the C₆₋₁₀ aromatic hydrocarbon include, but are not limited to, benzene, toluene, o-xylene, p-xylene, and m-xylene. In one specific embodiment, the C₆₋₁₀ aromatic hydrocarbon is toluene. Examples of the C₂₋₆ alkyl acetate include, but are not limited to, ethyl acetate and isobutyl acetate. Examples of the C₄₋₁₀ ether include, but are not limited to, linear and branched C₄₋₁₀ ether. The linear or branched C₄₋₁₀ ether is diethyl ether, or methyl tert-butyl ether. In one specific embodiment, the cyclic C₄₋₁₀ ether is tetrahydrofuran. Examples of the C₃₋₆ ketone include, but are not limited to, acetone, methyl ethyl ketone, and methyl iso-butyl ketone.

According to one specific example, the first solvent is toluene, and the second solvent is heptanes. In another example, the first solvent is tetrahydrofuran, and the second solvent is water.

Accordingly, another aspect of the present invention is to provide particles of an active ingredient produced by the present method.

Many of the attendant features and advantages of the present disclosure will becomes better understood with reference to the following detailed description considered in connection with the accompanying drawings.

In accordance with common practice, the various described features/elements are not drawn to scale but instead are drawn to best illustrate specific features/elements relevant to the present invention. Also, like reference numerals and designations in the various drawings are used to indicate like elements/parts.

### BRIEF DESCRIPTION OF THE DRAWINGS

The present description will be better understood from the following detailed description read in light of the accompanying drawings, where:

FIG. 1A is a Scanning Electron Microscope (SEM) image illustrating the crystalline of nanoparticles without milling; and

FIG. 1B is a Scanning Electron Microscope (SEM) image illustrating the crystalline of nanoparticles with milling.

### DESCRIPTION

The detailed description provided below is not intended to represent the only forms in which the present example may be constructed or utilized. The description sets forth the functions of the example and the sequence of steps for constructing and operating the example. However, the same or equivalent functions and sequences may be accomplished by different examples.

1. Definitions

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Also, unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

Unless otherwise required by context, it will be understood that singular terms shall include plural forms of the same and plural terms shall include the singular. Also, as used herein and in the claims, the terms “at least one” and “one or more” have the same meaning and include one, two, three, or more. Furthermore, the phrases “at least one of A, B, and C”, “at least one of A, B, or C” and “at least one of A, B and/or C,” as use throughout this specification and the appended claims, are intended to cover A alone, B alone, C alone, A and B together, B and C together, A and C together, as well as A, B, and C together.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about.” Accordingly, unless indicated to the contrary, the numerical parameters set forth in the present disclosure and attaching claims are approximations that can vary as desired. At the very least, each numerical parameter should at least be construed in light of the number of reported significant digits and by applying ordinary rounding techniques. Ranges can be expressed herein as from one endpoint to another endpoint or between two endpoints. All ranges disclosed herein are inclusive of the endpoints, unless specified otherwise.

The term “mixing” as used herein refers to the action of adding one solvent/solution to another solvent/solution, without been bound by any particular sequence of which solvent/solution is added first. In one example, a solvent is added to a solution comprising an active pharmaceutical ingredient (API) (e.g., paliperidone palmitate). In another example, the solution comprising the API is added to the solvent.

As used herein, an “excipient” is one that is suitable for use in a subject without adverse side effects (such as toxicity, irritation, and allergic response) while commensurate with a reasonable benefit/risk ratio. Also, each excipient must be “acceptable” in the sense of being compatible with the other ingredients of the particles of the present disclosure.

As used herein, the term “active ingredient” or “active pharmaceutical ingredient” refers to, physiologically active peptides, antitumor agents, antibiotics, anti-pyretic agents, analgesics, anti-inflammatory agents, antitussive expectorants, sedatives, muscle relaxants, anti-epileptics, anti-ulcer agents, anti-depressants, anti-allergic agents, cardiotonics, anti-arrhythmic agents, vasodilators, hypotensive diuretics, anti-diabetics, anti-hyperlipidemic agents, anti-coagulants, anti-oxidant, hemolytics, anti-tuberculosis agents, hormones, narcotic antagonists, bone resorption suppressors, osteogenesis promoters or angiogenesis inhibitors.

Examples of the physiologically active peptides may be selected from the group consisting of, growth hormone releasing peptide (GHRP), luteinizing hormone-releasing hormone (LHRH), bombesin, gastrin releasing peptide (GRP), calcitonin, bradykinin, galanin, melanocyte-stimulating hormone (MSH), growth hormone releasing factor (GRF), gonadotropin-releasing hormone agonist (GnRH agonist)(e.g., triptorelin pamoate, triptorelin acetate, buserelin, histrelin, goserelin, leuprolide, deslorelin, nafarelin, and triptorelin), amylin, tachykinins, secretin, parathyroid hormone (PTH), endothelin, calcitonin gene releasing peptide (CGRP), neuromedins, parathyroid hormone related protein (PTHrP), glucagon, adrenocorticothrophic hormone (ACTH), peptide YY (PYY), glucagon-like peptide-1 (GLP1), liraglutide, exenatide, lixisenatide, albiglutide, dulaglutide, taspoglutide, semaglutide, vasoactive intestinal peptide (VIP), pituitary adenylate cyclase activating peptide (PACAP), motilin, substance P, neuropeptide Y (NPY), thyroid stimulating hormone (TSH), insulin, somatostatin, growth hormones, prolactin, adrenocorticotropic hormone (ACTH), ACTH derivatives (e.g., ebiratide), thyrotropin-releasing hormone, thyroid-stimulating hormone (TSH), luteinizing hormone (LH), follicle-stimulating hormone (FSH), vasopressin, oxytocin, gastrin, pancreozymin, cholecystokinin, angiotensin, human placental lactogen, human chorionic gonadotropin (HCG), enkephalin, endorphin, kyotorphin, interferons, interleukins, tuftsin, thymopoietin, thymosin, thymostimulin, thymic humoral factor (THF), blood thymic factor (FTS), other thymic factors, tumor necrosis factor (TNF), colony-stimulating factors (e.g., CSF, GCSF, GMCSF, MCSF), dynorphin, neurotensin, caerulein, urokinase, asparaginase, kallikrein, insulin-like growth factors (IGF-I, IGF-II), nerve growth factor (NGF), cell growth factors (e.g., EGF, TGF-.alpha., TGF-.beta., PDGF, acidic FGF, basic FGF), bone morphogenic factor (BMP), nerve nutrition factors (e.g., NT-3, NT-4, CNTF, GDNF, BDNF), blood coagulation factors VIII and IX, lysozyme chloride, polymixin B, colistin, gramicidin, bacitracin, erythropoietin (EPO), thrombopoietin (TPO), and endothelin-antagonistic peptides and analogs, fragments, derivatives and salts thereof. In one preferred embodiment, the active ingredient(s) is LH-RH or an analog thereof, still more preferably leuprorelin or leuprorelin acetate. In one preferred embodiment, the active ingredient(s) is GLP-1 or an analog thereof, still more preferably exenatide.

Examples of the anti-tumor agents include, but are not limited to, bleomycin, methotrexate, actinomycin D, mitomycin C, binblastin sulfate, bincrystin sulfate, daunorubicin, adriamycin, neocartinostatin, cytosinearabinoside, fluorouracil, tetrahydrofuryl-5-fluorouracil, krestin, picibanil, lentinan, levamisole, bestatin, azimexon, glycyrrhizin, poly I:C (polyinosinic-polycytidylic acid), polyA:U (Polyadenylic-polyuridylic acid) and poly ICLC (Polyinosinic-Polycytidylic acid with Polylysine and Carboxymethylcellulose).

Examples of the antibiotics include, but are not limited to, gentamicin, dibekacin, kanendomycin, lividomycin, tobramycin, amikacin, fradiomycin, sisomycin, tetracycline hydrochloride, oxytetracycline hydrochloride, rolitetracycline, doxycycline hydrochloride, ampicillin, piperacillin, ticarcillin, cefalothin, cefaloridine, cefotiam, cefsulodin, cefmenoxime, cefmetazole, cefazolin, cefotaxime, cefoperazon, ceftizoxime, mochisalactam, thienamycin, sulfazecin and aztreonam.

Examples of the anti-pyretic agents, analgesics and anti-inflammatory agents include, but are not limited to, salicylic acid, sulpyrine, flufenamic acid, diclofenac, indomethacin, morphine, pethidine hydrochloride, levorphanol tartrate and oxymorphone.

Examples of the anti-tussive expectorants include, but are not limited to, ephedrine hydrochloride, methylephedrine hydrochloride, noscapine hydrochloride, codeine phosphate, dihydrocodeine phosphate, allocramide hydrochloride, clofedanol hydrochloride, picoperidamine hydrochloride, chloperastine, protokylol hydrochloride, isoproterenol hydrochloride, sulbutamol sulfate and terbutaline sulfate.

Examples of the sedatives include, but are not limited to, chlorpromazine, prochlorperazine, trifltioperazine, atropine sulfate and methylscopolamine bromide.

Examples of the muscle relaxants include, but are not limited to, pridinol methanesulfonate, tubocurarine chloride and pancuronium bromide.

Examples of the anti-epileptics include, but are not limited to, phenytoin, ethosuximide, acetazolamide sodium and chlordiazepoxide.

Examples of the anti-ulcer agents include, but are not limited to, metoclopramide and histidine hydrochloride.

Examples of the anti-depressants include, but are not limited to, imipramine, clomipramine, noxiptiline and phenerdine sulfate, amitriptyline HCl, amoxapine, butriptyline HCl, clomipramine HCl, desipramine HCl, dothiepin HCl, doxepin HCl, fluoxetine, gepirone, lithium carbonate, mianserin HCl, milnacipran, nortriptyline HCl and paroxetine HCl; anti-muscarinic agents such as atropine sulphate and hyoscine; sedating agents such as alprazolam, buspirone HCl, chlordiazepoxide HCl, chlorpromazine, clozapine, diazepam, flupenthixol HCl, fluphenazine, flurazepam, lorazepam, mazapertine, olanzapine, oxazepam, pimozide, pipamperone, piracetam, promazine, risperidone, paliperidone, paliperidone palmitate, selfotel, seroquel, sulpiride, temazepam, thiothixene, triazolam, trifluperidol and ziprasidone; anti-migraine drugs such as alniditan and sumatriptan; beta-adrenoreptor blocking agents such as atenolol, carvedilol, metoprolol, nebivolol and propranolol; anti-Parkinsonian drugs such as bromocryptine mesylate, levodopa and selegiline HCl; opioid analgesics such as buprenorphine HCl, codeine, dextromoramide and dihydrocodeine; parasympathomimetics such as galanthamine, neostigmine, physostymine, tacrine, donepezil, ENA 713 (exelon) and xanomeline; and vasodilators such as amlodipine, buflomedil, amyl nitrite, diltiazem, dipyridamole, glyceryl trinitrate, isosorbide dinitrate, lidoflazine, molsidomine, nicardipine, nifedipine, oxpentifylline and pentaerythritol tetranitrate.

Examples of the anti-allergic agents include, but are not limited to, diphenhydramine hydrochloride, chlorpheniramine maleate, tripelenamine hydrochloride, methdilazine hydrochloride, clemizole hydrochloride, diphenylpyraline hydrochloride and methoxyphenamine hydrochloride.

Examples of the cardiotonics include, but are not limited to, trans-paioxocamphor, theophyllol, aminophylline and etilefrine hydrochloride.

Examples of the anti-arrhythmic agents include propranol, alprenolol, bufetolol and oxprenolol.

Examples of the vasodilators include, but are not limited to, oxyfedrine hydrochloride, diltiazem, tolazoline hydrochloride, hexobendine and bamethan sulfate.

Examples of the hypotensive diuretics include, but are not limited to, hexamethonium bromide, pentolinium, mecamylamine hydrochloride, ecarazine hydrochloride and clonidine.

Examples of the anti-diabetics include, but are not limited to, glymidine sodium, glipizide, fenformin hydrochloride, buformin hydrochloride and metformin.

Examples of the anti-hyperlipidemic agents include, but are not limited to, pravastatin sodium, simvastatin, clinofibrate, clofibrate, simfibrate and bezafibrate.

Example of the anti-coagulant includes, but is not limited to, heparin sodium.

Examples of the hemolytics include, but are not limited to, thromboplastin, thrombin, menadione sodium hydrogen sulfite, acetomenaphthone, epsilon-aminocaproic acid, tranexamic acid, carbazochrome sodium sulfonate and adrenochrome monoaminoguanidine methanesulfonate.

Examples of the anti-tuberculosis agents include, but are not limited to, isoniazid, ethambutol and p-aminosalicylic acid.

Examples of the hormones include, but are not limited to, predonizolone, predonizolone sodium phosphate, dexamethasone sodium sulfate, betamethasone sodium phosphate, hexestrol phosphate, hexestrol acetate and methimazole.

Examples of the narcotic antagonists include, but are not limited to, levallorphan tartrate, nalorphine hydrochloride and naloxone hydrochloride.

Example of the bone resorption suppressor includes, but is not limited to, ipriflavone.

Examples of the osteogenesis promoters include, but are not limited to, polypeptides such as BMP, PTH, TGF-β and IGF-1, and (2R,4S)-(−)-N-[4-(diethoxyphosphorylmethyl)phenyl]-1,2,4,5-tetrahydro-4-methyl-7, 8-methylenedioxy-5-oxo-3-benzothiepine-2-carboxamide and 2-(3-pyridyl)-ethane-1,1-diphosphonic acid.

Examples of the angiogenesis suppressors include, but are not limited to, angiogenesis-suppressing steroid, fumagillin and fumagillol derivatives.

2. Description of preferred embodiments

The present invention discloses an improved method for producing particles of an active ingredient, particularly nanoparticles and/or microparticles of the active ingredient. The thus produced therapeutic particles are not only easier to handle, but also exhibits improved properties, such as improved solubility, stability, and pharmacokinetics.

The present method produces therapeutic particles by taking advantage in the differences between miscibilities or solubilities of the active ingredient in two miscible solvent systems, so that the active ingredient soluble in a first solvent system precipitates when a second solvent is introduced therein.

Accordingly, to produce particles of the active ingredient, the active ingredient is mixed with the first solvent until it reaches complete dissolution thereby forming a first solution. Then, a second solvent is introduced into the first solution so that particles of the active ingredient are precipitated out of the first solution and thereby forming an emulsified solution. According to preferred embodiments of the present disclosure, the first and second solvents are miscible with each other, while the second solvent is immiscible with the first solution.

Suitable first and second solvents that may be used in the present method are those with solvent polarity index to water ranges between 0.001 and 1.000; such as 0.001, 0.002, 0.003, 0.004, 0.005, 0.006, 0.007, 0.008, 0.009, 0.010 0.011, 0.021, 0.031, 0.041, 0.051, 0.061, 0.071, 0.081, 0.091, 0.101, 0.111, 0.121, 0.131, 0.141, 0.151, 0.161, 0.171, 0.181, 0.191, 0.201, 0.211, 0.221, 0.231, 0.241, 0.251, 0.261, 0.271, 0.281, 0.291, 0.301, 0.311, 0.321, 0.331, 0.341, 0.351, 0.361, 0.371, 0.381, 0.391, 0.401, 0.411, 0.421, 0.431, 0.441, 0.451, 0.461, 0.471, 0.481, 0.491, 0.501, 0.511, 0.521, 0.531, 0.541, 0.551, 0.561, 0.571, 0.581, 0.591, 0.601, 0.611, 0.621, 0.631, 0.641, 0.651, 0.661, 0.671, 0.681, 0.691, 0.701, 0.711, 0.721, 0.731, 0.741, 0.751, 0.761, 0.771, 0.781, 0.791, 0.801, 0.811, 0.821, 0.831, 0.841, 0.851, 0.861, 0.871, 0.881, 0.891, 0.901, 0.911, 0.921, 0.931, 0.941, 0.951, 0.961, 0.971, 0.981, 0.991 and 1.000. Preferably, the solvent polarity index of the first solvent is 0.050-0.4000, such as, 0.050, 0.055, 0.065, 0.075, 0.085, 0.095, 0.105, 0.115, 0.125, 0.135, 0.145, 0.155, 0.165, 0.175, 0.185, 0.195, 0.205, 0.215, 0.225, 0.235, 0.245, 0.255, 0.265, 0.275, 0.285, 0.295, 0.305, 0.315, 0.325, 0.335, 0.345, 0.355, 0.365, 0.375, 0.385, 0.395 and 0.040. More preferably, the solvent polarity index of the first solvent is 0.050-0.350, such as 0.050, 0.060, 0.070, 0.080, 0.090, 0.100, 0.110, 0.120, 0.130, 0.140, 0.150, 0.160, 0.170, 0.180, 0.190, 0.200, 0.210, 0.220, 0.230, 0.240, 0.250, 0.260, 0.270, 0.280, 0.290, 0.300, 0.310, 0.320, 0.330, 0.340 or 0.350.

Each of the first and second solvents (i.e., solvents respectively having solvent polarity index ranging between 0.001 and 1.0) may be selected from the group consisting of, C₅₋₁₂ alkane, C₆₋₁₀ aromatic hydrocarbon, C₂₋₆ alkyl acetate, C₄₋₁₀ ether, C₄₋₁₀ cyclic ether, C₃₋₆ ketone, acetonitrile, and water. Suitable examples of the C₅₋₁₂ alkane include, but are not limited to, pentane, isopentane, hexane, cyclohexane, heptane, heptanes, octane, nonane, and etc. The heptanes is a mixtire of heptane isomers. Suitable examples of the C₆₋₁₀ aromatic hydrocarbon include, but are not limited to, benzene, toluene, o-xylene, p-xylene, and m-xylene. Suitable examples of the C₂₋₆ alkyl acetate include, but are not limited to, ethyl acetate and isobutyl acetate. Suitable examples of the C₄₋₁₀ ether is linear or branched C₄₋₁₀ ether which include, but are not limited to, diethyl ether, methyl tert-butyl ether. Suitable examples of the C₄₋₁₀ cyclic ether is tetrahydrofuran. Suitable examples of the C₃₋₆ ketone include, but are not limited to, acetone, methyl ethyl ketone, and methyl iso-butyl ketone.

According to embodiments to produce the paliperidone palmitate particles of the present disclosure, among the solvents that were tested, only 2 solvents (i.e., toluene and tetrahydrofuran) may be used as the first solvent in the present disclosure. Further, among the solvents that were tested, only 3 solvents (i.e., water, heptanes, and hexane) are qualified as the second solvent in the present method. According to one preferred example, the first solvent is tetrahydrofuran and the second solvent is water. In another preferred example, the first solvent is toluene and the second solvent is heptanes or hexane.

According to optional embodiments, the emulsified solution described above is filtered and subsequently dried to produce powders of particles of the active ingredient. Each particles of the paliperidone palmitate produced by the method disclosed herein is about 0.1μm to 10μm in diameter. Thus, the particles have a narrow particle size distribution.

To identify suitable solvents for use in the present method, various types of solvents and combinations thereof were tested. Among them, only 3 solvents (i.e., methanol, ethanol, and tetrahydrofuran) are suitable for use as the first solvent in the present method, and only 2 solvents (i.e., water and heptanes) are qualified as the second solvent in the present method. According to one preferred example, the first solvent is tetrahydrofuran and the second solvent is heptanes. In another preferred example, the first solvent is methanol and the second solvent is water. In one specific example, each therapeutic particles (i.e., the L-asorbic acid particles) produce by the present method is 7.8 to 47.48μm in diameter. Compare with the L-asorbic acid of raw material, the L-asorbic acid particles provided by present method has a narrower particle size distribution.

Further, due to the relatively narrower size distribution, the particles produced by the present invention exhibit improved properties that reflect on solubility, stability, and/or pharmacokinetics, as compared to those that rare relatively larger in size.

As could be appreciated, the particles of the present invention may be formulated into a pharmaceutical composition with pharmaceutically acceptable carriers, and can be administered to a subject orally or parenterally in various dosage forms. Parenteral administration includes, for example, administration by intraveneous, subcutaneous, intramuscular, transdermal, intrarectal, transnasal, and instillation methods.

The dosage form of the pharmaceutical composition for oral administration includes, for example, tablets, pills, granules, powders, solutions, suspensions, syrups or capsules. As a method of producing a tablet, a pill, granule or powder, it can be formed by conventional techniques using a pharmaceutically acceptable carrier such as excipient, binder, or disintegrant and etc. As to the form of a solution, suspension or syrup, it can be produced by conventional techniques using glycerol esters, alcohols, water or vegetable oils, and etc. The form of capsule can be produced by filling a capsule made of gelatin with the granule, powder or a solution prepared as described above. Among the agents for parenteral administration, in the case of intravenous, subcutaneous or intramuscular administration, it can be administered as injection. An injection can be prepared by dissolving the nanoparticles and/or microparticles of the present invention in water soluble solution such as physiological saline, or water insoluble solution consisting of organic esters such as propylene glycol, polyethylene glycol, or vegetable oils (e.g., sesame oil). In the case of transdermal administration, for example, a dosage form as an ointment or a cream can be employed. The ointment can be produced by mixing the nanoparticle and/or microparticles of the present invention with fats or oils and etc; and the cream can be produced by mixing the nanoparticle and/or microparticles of the present invention with emulsifiers. In the case of rectal administration, it may be in the form of suppository using a gelatin soft capsule. In the case of transdermal administration, it may be in a form of a liquid or a powdery formulation. In a liquid formulation, water, salt solution, phosphate buffer, acetate buffer and the like may be used as a base; it may also contain surfactants, antioxidants, stabilizers, preservatives or tackifiers. In a powdery formulation, it may contain water-absorbing materials such as water-soluble polyacrylates, cellulose low-alkyl esters, polyethylene glycol polyvinyl pyrrolidone, amylase, etc., and non-water absorbing materials such as cellulose, starches, gums, vegetable oils or cross-linked polymers. Further, antioxidants, colorants, preservatives may be added to the powdery formulation. The liquid or powdery formulation may be administered by use of a spray apparatus. In case of inhalation through nose or mouth, a solution or suspension containing the particle of the present disclosure and a pharmaceutical excipient generally accepted for this purpose is inhaled through an inhalant aerosol spray. Alternatively, the particle of the present disclosure in the form of a powder may be administered through inhalator that allows direct contact of the powder with the lung. To these formulations, if necessary, pharmaceutically acceptable carriers such as isotonic agents, preservatives, dispersions, or stabilizers may be added. Further, if necessary, these formulations may be sterilized by filtration, or by treatment with heat or irradiation.

The following examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### EXAMPLES

### Example 1

### Production and Characterization of Paliperidone Palmitate Particles

1.1 The Solubility Test of Paliperidone Palmitate in Various Types of Solvents

The solubility of paliperidone palmitate in various types of solvents was evaluated. To this purpose, paliperidone palmitate and the designated solvent were mixed in a ratio of 1/15 (v/w) at 30° C. and the mixture was stirred for about 0.5 hour. Results are summarized in Table 1.

TABLE 1
Solubility of paliperidone palmitate in various types of solvents

| | | Solubility of paliperidone |
|---|---|---|
| First Solvent | Solvent Polarity Index | palmitate |
| Water | 1.000 | insoluble |
| EtOH | 0.654 | insoluble |
| IPA | 0.546 | insoluble |
| ACN | 0.460 | insoluble |
| Acetone | 0.355 | insoluble |
| DCM | 0.309 | soluble |
| EtOAc | 0.028 | insoluble |
| THF | 0.207 | soluble |
| MTBE | 0.124 | insoluble |
| Toluene | 0.099 | soluble |
| Heptanes | 0.012 | insoluble |
| Hexane | 0.009 | insoluble |

ACN: Acetonitrile;
DCM: Dichloromethane;
EtOAc: Ethyl acetate;
EtOH: ethanol;
IPA: Isopropyl alcohol;
MTBE: Methyl tert-butyl ether;
THF: tetrahydrofuran

Among the 12 solvents that were tested, paliperidone palmitate was found to be soluble only in DCM, THF, and toluene, and insoluble in the rest of solvents. Thus, DCM, THF and toluene were independently chosen as the first solvent for subsequent production process.

1.2 Emulsion Test of Paliperidone Palmitate in Various Types of Solvents

Paliperidone palmitate was dissolved in DCM, THF, or toluene at 30° C. and stirred for about 0.5 hour to form a first solution, then a second solvent (as indicated in Table 2) was added and stirred for 3 hr. As the data in Table 2 indicated, among the 36 combinations (i.e., the combination of one of the 3 first solutions and one of the second solvents), only three combinations gave satisfied emulsified solutions, which comprised particles of paliperidone palmitate. The 3 combinations were THF/water, toluene/heptanes, and toluene/hexane, respectively.

TABLE 2
Solubility of the second solvent in the first
solution containing paliperidone palmitate

Paliperidone palmitate
dissolved in the first solvent

| The Second solvent | DCM | THF | Toluene |
|---|---|---|---|
| Water | layered | emulsified | layered |
| EtOH | miscible | miscible | miscible |
| IPA | miscible | miscible | miscible |
| ACN | miscible | miscible | miscible |
| Acetone | miscible | miscible | miscible |
| DCM | — | miscible | miscible |
| EtOAc | miscible | miscible | miscible |
| THF | miscible | — | miscible |
| MTBE | miscible | miscible | miscible |
| Toluene | miscible | miscible | — |
| Heptanes | miscible | miscible | emulsified |
| Hexane | miscible | miscible | emulsified |

ACN: Acetonitrile;
DCM: Dichloromethane;
EtOAc: Ethyl acetate;
EtOH: ethanol;
IPA: Isopropyl alcohol;
MTBE: Methyl tert-butyl ether;
THF: tetrahydrofuran

1.3 Production of the Particles of Paliperidone Palmitate Using Toluene and Heptanes

1.3.1 Process A

Seven batches of paliperidone palmitate particles were respectively prepared in accordance to conditions indicated in Tables 3 and 4. Take batch 2 as an example, a 5-L reactor equipped with a mechanical stirrer was charged with paliperidone palmitate and toluene at the first temperature (i.e., 30° C.) and stirred at the first speed (i.e., 293 rpm) until paliperidone palmitate was completely dissolved in toluene, thereby forming a clear solution. Then, heptanes was added into the clear solution (temperature was approximately 25° C.) and stirred at the second speed (i.e., 326 rpm) to form an emulsified solution. The emulsified solution was further cooled to the second temperature (i.e., 0 to 5° C.), and stirred for additional 3 hours until paliperidone palmitate particles were formed. Then, the particles were collected and rinsed with cold heptanes (i.e., 0 to 5° C.) and dried at ambient temperature under vacuum.

TABLE 3
Conditions for the production of paliperidone palmitate particles of batches 1 to 4

| | Batch No. | | | | | | | |
|---|---|---|---|---|---|---|---|---|
| | 1 | | 2 | | 3 | | 4 | |
| Reactor | 5 | L | 5 | L | 5 | L | 5 | L |
| Crude API | 80.0 | g | 80.0 | g | 80.0 | g | 80.0 | g |
| Toluene | 1040.8 | g | 1040.8 | g | 1041 | g | 1068.8 | g |
| Heptanes | 888.02 | g | 888.02 | g | 888.0 | g | 911.9 | g** |
| 1^{st} speed (rpm) | 307 | | 293 | | 294 | | 300 | |
| 1^{st} Temperature | 30 | | 30 | | 30 | | 32 | |
| 2^{nd} speed (rpm) | 335 | | 326 | | 345 | | 318 | |
| 2^{nd} Temperature | 0-5° | C. | 0-5° | C. | 0-5° | C. | 0-5° | C. |

| | | | | |
|---|---|---|---|---|
| Concentration | 0.0769 | 0.0769 | 0.0768 | 0.0769 |
| (Crude API | | | | |
| g/toluene g) | | | | |
| Weight | 1.171 | 1.171 | 1.171 | 1.172 |
| Ratio of | | | | |
| toluene to | | | | |
| heptanes | | | | |

**The heptanes is pre-heated to 30° C.

TABLE 4
Conditions for the production of paliperidone
palmitate particles of batches 5 to 7

| Batch No. | | | |
|---|---|---|---|
| | 5 | 6 | 7 |

| Reactor | 1 | L | 1 | L | 1 | L |
|---|---|---|---|---|---|---|
| Crude API | 20.1 | g | 20.1 | g | 20.2 | g |
| Toluene | 261.5 | g | 261.5 | g | 262.2 | g |
| Heptanes | 223.2 | g* | 223.2 | g* | 223.7 | g* |
| 1^{st} speed (rpm) | 160 | | 165 | | 186 | |
| 1^{st} Temperature | 37 | | 38 | | 42 | |
| 2^{nd} speed (rpm) | 160 | | 165 | | 171 | |
| 2^{nd} Temperature | 0-5° | C. | 0-5° | C. | 0-5° | C. |

| | | | |
|---|---|---|---|
| Concentration | 0.0769 | 0.0769 | 0.077 |
| (Crude API | | | |
| g/toluene g) | | | |
| Weight | 1.171 | 1.171 | 1.172 |
| Ratio of | | | |
| toluene to | | | |
| Heptanes | | | |

*The heptanes is pre-chilled to 0° C.

1.3.2

Process B

In this example, heptanes was charged into the reactor first, then the clear solution of API (e.g., paliperidone palmitate) and toluene was added to the heptanes within the reactor. Four batches of paliperidone palmitate particles were respectively prepared in accordance to conditions designated in Tables 5. Take batch 8 as an example, paliperidone palmitate was added into toluene at the first temperature (i.e., 30° C.), and stirred at the first speed (i.e., 169 rpm) until paliperidone palmitate was completely dissolved in toluene, thereby forming a clear solution. A 1-L reactor equipped with a mechanical stirrer was charged with heptanes, and the clear solution (temperature was approximately 25° C.) was added into the reactor and stirred at the second speed (i.e., 169 rpm) to form an emulsified solution. The emulsified solution was further cooled to the second temperature (i.e., 0 to 5° C.), and stirred for additional 3 hours until paliperidone palmitate particles were formed. Then, the particles were collected and rinsed with cold heptanes (i.e., 0 to 5° C.) and dried at ambient temperature under vacuum.

TABLE 5
Conditions for the production of paliperidone
palmitate particles of batches 8 to 10

| Batch No. | | | |
|---|---|---|---|
| | 8 | 9 | 10 |

| Reactor | 1 | L | 1 | L | 1 | L |
|---|---|---|---|---|---|---|
| Crude API | 20.3 | g | 20.3 | g | 20.3 | g |
| Toluene | 264.9 | g | 264.9 | g | 264.9 | g |
| Heptanes | 226.0 | g* | 226.0 | g* | 226.0 | g* |
| 1^{st} speed (rpm) | 169 | | 170 | | 172 | |
| 1^{st} Temperature | 40 | | 40 | | 40 | |
| 2^{nd} speed | 169 | | 170 | | 170 | |
| (rpm) | | | | | | |
| 2^{nd} Temperature | 0-5° | C. | 0-5° | C. | 0-5° | C. |

| | | | |
|---|---|---|---|
| Concentration | 0.0769 | 0.077 | 0.077 |
| (Crude API | | | |
| g/toluene g) | | | |
| Weight | 1.171 | 1.172 | 1.172 |
| Ratio of | | | |
| toluene to | | | |
| Heptanes | | | |

*The heptanes was pre-chilled to 0° C.

1.4 Production of Paliperidone Palmitate Particles Using Xylene and Heptanes

The batches 11 to 12 of paliperidone palmitate particles were prepared in accordance to conditions indicated in Table 6. Take batch 11 as an example, a 1-L reactor equipped with a mechanical stirrer was charged with paliperidone palmitate and p-xylene at the first temperature (i.e., 40° C.) and stirred at the first speed (i.e., 176 rpm) until paliperidone palmitate was completely dissolved in p-xylene, thereby forming a clear solution. Then, heptanes was added into the clear solution (temperature was approximately 25° C.) within the reactor and stirred at the second speed (i.e., 176 rpm) to form an emulsified solution. The emulsified solution was further cooled to the second temperature (i.e., 0 to 5° C.), and stirred for additional 3 hours until paliperidone palmitate particles were formed. Then, the particles were collected and rinsed with cold heptanes (0 to 5° C.) and dried at ambient temperature under vacuum.

TABLE 6
Conditions for the production of paliperidone
palmitate particles of batches 11 and 12

| Batch No. | | |
|---|---|---|
| | 11 | 12 |

| Reactor | 1 | L | 1 | L |
|---|---|---|---|---|
| Crude API | 23 | g | 19.5 | g |
| p-Xylene | 344.1 | g | | |
| o-Xylene | | | 291.6 | g |
| Heptanes | 254.8 | g | 215.9 | g |
| 1^{st} speed (rpm) | 176 | | 176 | |
| 1^{st} Temperature | 40 | | 40 | |
| 2^{nd} speed (rpm) | 176 | | 176 | |
| 2^{nd} Temperature | 0-5° | C. | 0-5° | C. |

| | | |
|---|---|---|
| Concentration | 0.0668 | 0.669 |
| (Crude API | | |
| g/Xylene g) | | |
| Weight Ratio | 1.35 | 1.35 |
| of Xylene to | | |
| Heptanes | | |

1.5 Production of the Particles of Paliperidone Palmitate Using Toluene and Hexane

In this example, another batch 13 of paliperidone palmitate particles was prepared in accordance to conditions indicated in Table 7. A 1-L reactor equipped with a mechanical stirrer was charged with paliperidone palmitate and toluene at the first temperature (i.e., 38° C.) and stirred at the first speed (i.e., 160 rpm) until paliperidone palmitate was completely dissolved in toluene, thereby forming a clear solution. Then, n-hexane was added into the clear solution (temperature was approximately 25° C.) and stirred at the second speed (i.e., 178 rpm) to form an emulsified solution. The emulsified solution was further cooled to the second temperature (i.e., 0 to 5° C.), and stirred for additional 3 hours until paliperidone palmitate particles were formed. Then, the particles were collected and rinsed with cold n-hexane (0 to 5° C.) and dried at ambient temperature under vacuum.

TABLE 7
Conditions for the production of paliperidone
palmitate particles of batch 15

| | Batch No. | |
|---|---|---|
| | 13 | |
| Reactor | 1 | L |
| Crude API | 29.9 | g |
| Toluene | 389.1 | g |
| n-Hexane | 285.5 | g |
| 1^{st} speed (rpm) | 160 | |
| 1^{st} Temperature | 68 | |
| 2^{nd} speed (rpm) | 178 | |
| 2^{nd} Temperature | 0-5° | C. |

| | |
|---|---|
| Concentration | 0.0769 |
| (Crude API | |
| g/toluene g) | |
| Weight | 1.171 |
| Ratio of | |
| toluene to | |
| n-Hexane | |

*The n-hexane is pre-chilled to 0° C.

1.6 Characterization of the Particles of Examples 1.1 to 1.5

The paliperidone palmitate particles of Examples 1.1 to 1.5 were subject to particle diameter distribution analysis using a dynamic light scattering particle size distribution analyzer, and the particle size distribution was expressed as D10, D50 and D90. D10, D50 and D90 respectively represent the value of the particle diameter at 10%, 50% and 90% of the particles size distribution. The particle size distribution of paliperidone palmitate particles was as follows: D10: 1.00 to 2.51μm; D 50: 1.67 to 5.05μm; D 90: 3.27 to 9.79μm, and the yield of each size distribution was about 75.5% to 92.44% (see, Table 8 below). The results demonstrated that the particles produced by the present method had a small particle size and a narrow particle size distribution, as compared to that of the control. Further, the present method did not result in the loss of yields, as yields in general remained to be over 75-92%.

TABLE 8
Particle size distribution of paliperidone
palmitate particles of batches 1 to 13

| Batch No. of | | | | |
|---|---|---|---|---|
| the sample | D10 (μm) | D50 (μm) | D90 (μm) | Yield |
| 1 | 1.92 | 3.41 | 6.30 | 84.4% |
| 2 | 1.74 | 3.39 | 6.07 | 80.6% |
| 3 | 1.24 | 3.23 | 6.49 | 82.0% |
| 4 | 1.59 | 3.15 | 5.91 | 92.44 |
| 5 | 1.00 | 1.67 | 3.27 | 75.5% |
| 6 | 1.20 | 2.20 | 4.94 | 76.4% |
| 7 | 1.47 | 3.17 | 6.45 | 85.2% |
| 8 | 1.35 | 2.88 | 7.58 | 86.5% |
| 9 | 2.38 | 5.05 | 9.79 | 89.0% |
| 10 | 1.88 | 4.16 | 7.30 | 90.48% |
| 11 | 2.51 | 4.20 | 6.69 | 92.42% |
| 12 | 0.24 | 2.93 | 6.45 | 90.23% |
| 13 | 1.59 | 3.15 | 5.91 | 92.44% |

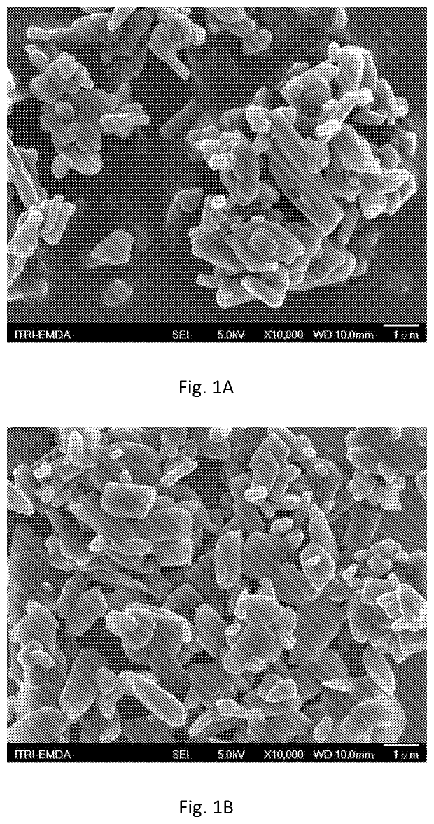

Furthermore, the crystalline morphology of the paliperidone palmitate particles produced by the present method were further subject to grinding, and the Scanning Electron Microscope (SEM) images taken before and after grinding indicated that grinding did not affect the crystalline structure of the present paliperidone palmitate particles (FIG. 1A and FIG. 1B), which were all in rectangular-shaped. In addition, it was found that the present paliperidone palmitate particles were easier to grind, as compared to the raw paliperidone palmitate particles (i.e., without being treated by the present method).

### Example 2

### Production and Characterization of L-ascorbic acid microparticles

2.1 The Solubility Test of L-ascorbic Acid in Various Types of Solvents

The solubility of L-ascorbic acid in various types of solvents was evaluated. To this purpose, L-ascorbic acid and the designated solvent were mixed in a ratio of 1/15 (v/w) at 30° C. and stirred for about 0.5 hour. Results are summarized in Table 9.

TABLE 9
Solubility of L-ascorbic acid in various types of solvents

| | Solubility of L-ascorbic |
|---|---|
| First Solvent | acid |
| MeOH | Soluble |
| EtOH | Soluble |
| IPA | insoluble |
| ACN | insoluble |
| Acetone | insoluble |
| DCM | insoluble |
| THF | soluble |
| Water | insoluble |
| Toluene | insoluble |
| Heptanes | insoluble |

MeOH: methanol;
EtOH: ethanol;
IPA: Isopropyl alcohol;
ACN: Acetonitrile;
DCM: Dichloromethane;
THF: tetrahydrofuran;
EtOAc: Ethyl acetate

Among the 10 solvents that were tested, L-ascorbic acid was found to be soluble only in methanol, ethanol, and tetrahydrofuran, and insoluble in the rest of solvents. Thus, methanol, ethanol, and tetrahydrofuran were independently chosen as the first solvent for subsequent production process.

2.2 Emulsion test of L-ascorbic Acid in Various Types of Solvents

L-ascorbic acid was dissolved in methanol or tetrahydrofuran at 30° C. and stirred for about 0.5 hour to form a first solution, then a second solvent (as indicated in Table 9) was added and stirred for 3 hr. Two combinations of solvents gave emulsified solutions, there were tetrahydrofuran/heptanes and methanol/water

2.3 Production of the L-ascorbic Acid Particles Using Methanol and Water

In this example, batch 14 of L-ascorbic acid was prepared. Briefly, a 1-L reactor equipped with a mechanical stirrer was charged with L-ascorbic acid (19.96 g) and methanol (144.8 g) at the first temperature (i.e., 40° C.) and stirred at the first speed (i.e., 190 rpm) until L-ascorbic acid was completely dissolved in methanol, thereby forming a clear solution. Then, cold water (i.e., 0° C.) was added into the clear solution (temperature was approximately 25° C.) and stirred at the second speed (i.e., 185 rpm) to form an emulsified solution. The emulsified solution was further cooled to the second temperature (i.e., 0 to 5° C.), and stirred for additional 3 hours until L-ascorbic acid particles were formed. Then, the particles were collected and rinsed with cold water (i.e., 0 to 5° C.) and dried at ambient temperature under vacuum.

2.4 Production of the L-ascorbic Acid Particles Using Tetrahydrofuran and Heptanes

Batch 15 of L-ascorbic acid was prepared as following. In this example, batch 17 of L-ascorbic acid was prepared. Briefly, a 1-L reactor equipped with a mechanical stirrer was charged with L-ascorbic acid (3.98 g) and tetrahydrofuran (65.1 g) at the first temperature (i.e., 40° C.) and stirred at the first speed (i.e., 190 rpm) until L-ascorbic acid was completely dissolved in tetrahydrofuran, thereby forming a clear solution. Then, cold heptanes (i.e., 0° C.) was added into the clear solution (temperature was approximately 25° C.) and stirred at the second speed (i.e., 185 rpm) to form an emulsified solution. The emulsified solution was further cooled to the second temperature (i.e., 0 to 5° C.), and stirred for additional 30 min until L-ascorbic acid particles were formed. Then, the particles were collected and rinsed with cold heptanes (i.e., 0 to 5° C.) and dried at ambient temperature under vacuum.

2.5 Characterization of the Particles of Examples 2.3 to 2.4

The L-ascorbic acid particles of Examples 2.3 to 2.4 were subject to particle diameter distribution analysis using a dynamic light scattering particle size distribution analyzer, and the particle size distribution was expressed as D10, D50 and D90. The particle size distributions of L-ascorbic acid particles of example 2.4 were as follows: D10: 7.8 to 9.3μm; D 50: 14.86 to 15.99μm; D 90: 39.34 to 47.48μm, and the yield of each size distributions was about 97.5% to 99.2% (see, Table 10 below). It is evident that the particles produced by the present method were relatively smaller in size and exhibited a narrower particle size distribution, as compared to those of L-ascorbic acid of raw material (i.e., the control). Further, the present method did not result in the loss of yields, as yields remained to be over 97%, even over 99%.

TABLE 10
Particle size distribution of L-ascorbic
acid particles of batches 14 and 15

| Batch No. of | | | | |
|---|---|---|---|---|
| the sample | D10 (μm) | D50 (μm) | D90 (μm) | Yield |
| 14 | 9.39 | 15.99 | 39.34 | 99.2% |
| 15 | 7.80 | 14.86 | 47.48 | 97.5% |
| Control | 8.70 | 15.00 | 68.2 | — |

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A method for producing particles of an active ingredient comprising:
(a) forming a first solution by dissolving the active ingredient in a first solvent; and
(b) forming the particles of the active ingredient by mixing a second solvent with the first solution to produce an emulsified solution;
wherein, the second solvent is miscible with the first solvent, but is immiscible with the first solution.

2. The method of the claim 1, wherein the active ingredient has a melting point, which is above 0° C.

3. The method of the claim 1, wherein the active ingredient is selected from the group consisting of, a physiologically active peptide, an antitumor agent, an antibiotic, an anti-pyretic agent, an analgesic, an anti-inflammatory agent, an antitussive expectorant, a sedative, a muscle relaxant, an anti-epileptic, an anti-ulcer agent, an anti-depressant, an anti-allergic agent, a cardiotonic, an anti-arrhythmic agent, a vasodilator, a hypotensive diuretic, an anti-diabetic, an anti-hyperlipidemic agent, an anti-coagulant, an anti-oxidant, a hemolytic, an anti-tuberculosis agent, a hormone, a narcotic antagonist, a bone resorption suppressor, an osteogenesis promoter, and an angiogenesis inhibitor.

4. The method of the claim 1, further comprising:
(c) removing the first and second solvents from the emulsified solution to produce a powder of the particles of the active ingredient.

5. The method of the claim 1, further comprising:
(a-1) filtering the first solution before the step (b).

6. The method of the claim 5, wherein the first solution is filtered through a filter having a pore size of 0.22

7. The method of the claim 1, wherein the first and second solvents are independently selected from the group consisting of, C₅₋₁₂ alkane, C₆₋₁₀ aromatic hydrocarbon, C₂₋₆ alkyl acetate, C₄₋₁₀ ether, C₄₋₁₀ cyclic ether, C₃₋₆ ketone, acetonitrile, and water.

8. The method of the claim 7, wherein the C₅₋₁₂ alkane is cyclohexane, hexane, heptane, heptanes, or octane.

9. The method of claim 8, wherein the C₅₋₁₂ alkane is heptane or heptanes.

10. The method of the claim 7, wherein the C₆₋₁₀ aromatic hydrocarbon is benzene, toluene, o-xylene, p-xylene, or m-xylene.

11. The method of the claim 10, wherein the C₆₋₁₀ aromatic hydrocarbon is toluene.

12. The method of the claim 7, wherein the C₂₋₆ alkyl acetate is ethyl acetate or isobutyl acetate.

13. The method of the claim 7, wherein the first solvent is C₄₋₁₀ ether or C₄₋₁₀ cyclic ether, and the second solvent is water.

14. The method of the claim 13, wherein the C₄₋₁₀ ether is diethyl ether or methyl tert-butyl ether.

15. The method of the claim 13, wherein the C₄₋₁₀ cyclic ether is tetrahydrofuran.

16. The method of the claim 7, wherein the C₃₋₆ ketone is selected from the group consisting of acetone, methyl ethyl ketone, and methyl iso-butyl ketone.

17. The method of the claim 15, wherein the first solvent is tetrahydrofuran, and the second solvent is water.
